(19) United States
(12) Patent Application Publication
(10) Pub. No.: US 2026/0243637 A1
(43) Pub. Date: Aug. 20, 2026

(54) SENSOR APPARATUS FOR PARTIALLY SATURATED POROUS MEDIA APPLICATIONS

(71) Applicant: COMMONWEALTH SCIENTIFIC AND INDUSTRIAL RESEARCH ORGANISATION, Australian Capital Territory (AU)

(72) Inventors: Miao Chen, Victoria (AU); David John Molenaar, Victoria (AU); Warren John Bruckard, Victoria (AU)

(21) Appl. No.: 19/105,941

(22) PCT Filed: Aug. 25, 2023
(86) PCT No.: PCT/AU23/50825

§ 371 Date: Feb. 24, 2025

(30) Foreign Application Priority Data
Aug. 26, 2022 (AU) 2022902445

(51) Int. Cl. G01N 1/40; B01D 29/56; G01N 27/06; G01N 27/416

(52) U.S. Cl. CPC G01N 1/4077; B01D 29/56; G01N 27/06; G01N 27/4167; G01N 2001/4088

(57) ABSTRACT

The present disclosure relates to a sensor apparatus 10 locatable within a region of porous media 72 for acquiring data from a liquid L within the porous media 72. The apparatus 10 includes a sensor housing 14 including an inlet 18 for receiving liquid L from the porous media 72 into the sensor housing 14 and an outlet 34 for received liquid L to egress the sensor housing 14. The apparatus 10 is configured for received liquid L to flow through the sensor housing 14 along a predetermined flow path between the inlet 18 and the outlet 34. The sensor apparatus 10 further includes a sensor arrangement 38 within the sensor housing 14 for acquiring data from the flow of liquid L between the inlet 14 and the outlet 34. The sensor arrangement 38 includes one or more sensors positioned along the flow path for contacting the liquid L and configured for measuring one or more properties of the liquid L to acquire data representative of the liquid L at the porous media region 72. The sensor apparatus 10 further includes a filtration arrangement 86 configured for removing particulates from the liquid L prior to contacting the one or more sensors.

### PRIORITY CROSS-REFERENCE

The present application claims priority to Australian provisional patent application 2022902445 filed 26 Aug. 2022, the entire contents of which is incorporated herein by reference.

### TECHNICAL FIELD

The present application relates to the in-situ and continuous monitoring of physical and/or chemical parameters of liquids within porous media of industrial, environmental and experimental processes. More particularly, the invention relates to a sensor apparatus for monitoring physical and/or chemical parameters of a liquid in an unsaturated or partially saturated porous media environment such as in the areas of heap leaching, in-situ leaching, dump leaching, soil remediation, materials/energy recycling processes and migration and remediation of aqueous phases in sub-ground surface environments. The invention also relates to apparatus in laboratories which are used to simulate the above environment and conditions, for example a laboratory column simulating heap leaching.

### BACKGROUND OF INVENTION

The following discussion of the background to the invention is intended to facilitate an understanding of the invention. However, it should be appreciated that the discussion is not an acknowledgement or admission that any aspect of the discussion was part of the common general knowledge as at the priority date of the application.

Various experimental and industrial processes require monitoring of liquids within porous media. An example of one such industrial process is that of heap leaching wherein a solvent liquid (typically an acid) is passed through a heap of crushed ore in order for the liquid to liberate/dissolve target minerals from the ore and is collected as a ‘pregnant’ or concentrated leaching solution (PLS) at the base of the heap for further processing to isolate/extract the target metal(s).

Another example of porous media liquid monitoring is that of column leaching which relates to an experimental apparatus consisting of a vertical column containing porous media and wherein a liquid lixiviant is irrigated onto the top of the column and passed through the media, again for collection at the base of the column for subsequent analyses and recycling to measure or determine the extent of interaction between the media and the liquid. Column leaching has broad applications including use as a test system for heap leaching applications as well as municipal water testing applications. Other example applications requiring monitoring of liquid with porous media include tank and vat leaching, mine waste remediation and soil remediation.

A problem with existing methods of porous media liquid testing and analyses is that testing of the sample liquid is typically performed ex-situ after the liquid is extracted or collected from the media. For example, sample liquid may be collected from within an ore heap and drawn to the surface for testing. Alternatively, liquid may be collected at the base of a heap (or a column leaching apparatus) and then transported to a testing laboratory at a different part of the facility or may even be offsite.

The resulting delay between sampling and analysis introduces inaccuracies insofar as the chemical and physical properties of the sample liquid can change between sampling and testing. For example, oxygen reduction potential (redox) is one time-sensitive parameter whereby the redox level of a sample in-situ can vary significantly from the redox level measured once the sample is removed from the media and exposed to oxygen or air for a period of time before analysis.

Furthermore, ex-situ and off-site laboratory analysis takes time, and there is a lag time between sampling, analysis and taking action which does not support prompt responsiveness to environmental or process issues. In some instances, the act of extracting liquid from the porous media can interfere with the process parameters (for example, the digging or introduction of a sampling pathway or

### SUMMARY OF INVENTION

It is therefore desirable to provide an alternative sensor apparatus for porous media applications which addresses at least some of the above-noted drawbacks with existing systems or which provides an alternative choice for operators of liquid monitoring and testing systems.

According to an aspect of the invention, there is provided a sensor apparatus locatable within a region of porous media for acquiring data from a liquid within the porous media, the apparatus including: a sensor housing including an inlet for receiving liquid from the porous media into the sensor housing and an outlet for received liquid to egress the sensor housing, the apparatus configured for received liquid to flow through the sensor housing along a predetermined flow path between the inlet and the outlet; a sensor arrangement within the sensor housing for acquiring data from the flow of liquid between the inlet and the outlet, the sensor arrangement including one or more sensors positioned at the flow path for contacting the liquid and configured for measuring one or more properties of the liquid to acquire data representative of the liquid at the porous media region; and a filtration arrangement configured for removing particulates from the liquid prior to contacting the one or more sensors.

The present invention advantageously provides a sensor apparatus configured for location within a region of porous media to be analysed and such that data about the liquid in that region can be acquired in-situ and in real time. As compared to existing systems and apparatuses which test samples after extraction from porous media, the invention provides operators with more up to date information about the chemical and physical properties of liquid within a region of interest. Furthermore, the invention can reduce the lag time between sampling and data acquisition such that the data acquired is more representative of the liquid within the media and there is less opportunity for the chemical or physical properties of the sample to change between sampling and testing.

The invention may therefore allow for time continuous monitoring of liquid within a porous media without the need to periodically obtain samples and transport the samples to a testing laboratory. For example, the invention may allow for data to be acquired from liquid within the porous media within seconds of entering the sensor housing inlet and for the data to be transmitted to an appropriate receiving hub. The reduced lag time may provide information that is more actionable and in certain applications such as industrial processes may enable operators to respond more promptly to changing conditions within the porous media. The invention may thereby also facilitate closed loop process control.

The invention advantageously allows for liquid received through the inlet to flow through the sensor housing between the inlet and the outlet and for measurements of the liquid to occur between the inlet and the outlet. At the outlet, the received liquid may typically be returned to the porous media and continue its natural flow path. In this regard, the invention is advantageously configured so as not to interfere or disturb the bulk flow field and to representatively sample and measure the liquid passing through the sensor housing and for the measured liquid to be returned to the region of porous media. A sensor apparatus according to the invention may also be sized relatively small to minimise interference within the flow field. For example, in a particular embodiment, the sensor housing has a width of 45 mm and a length of 80 mm.

The porous media suitable for use with the present invention may be any porous material through which liquid flows and for which analysis of the liquid is desired. Depending on application, examples of possible porous media may be mined ore, mined ore waste, crushed and/or agglomerated mine ore, soil, dirt, rock, industrial particulates, industrial waste including e-waste, coal, clay or ash/fly ash.

The invention may have particular application in a heap leach or a system for simulating a heap leach such as a crib or column leaching apparatus. It will be appreciated that the heap leaching process involves crushed ore being placed on a lined pad which is then irrigated with a lixiviant to provide leachate which is then collected in a pond or tank. Depending on the ore, the location and type of the heap, the environmental conditions and the like, a number of parameters within the heap leach will dictate the effectiveness of the heap leach, the rate of metal dissolution, the amount of consumables used in the heap leach and the like. Cribs and column arrangements are utilised to simulate and model large scale heap leach applications in laboratory settings mostly. Presently, analysis of cribs and column arrangements are limited to determining parameters after the leachate has been extracted and collected at the bottom of the crib or column. This approach provides only limited or no insight into the operation of the heap leach in-situ and in real time. Thus, the invention may allow for both spatial and temporal insight into the heap operation in real-time.

The present invention advantageously enables for in-situ data to be acquired from a heap or from a crib or column leaching apparatus prior to leachate being collected from the bottom of the column or the heap. The invention may therefore provide operators with insights that may inform adjustment of one or more parameters of the heap leach to improve operation. For example, chemical properties of a fluid within a region of material over time may be monitored, which may inform adjustment of chemical parameters to improve extraction of metals from ore. Similarly, data acquired from in-situ measurement of liquid within a heap or column apparatus may inform optimisation of physical parameters such as irrigation drip rate, oxidation flow rate and the like.

The present invention may allow for continuous monitoring of bioleaching processes and in some applications may enable spatial monitoring of bioleaching heaps. For example, a sensor apparatus of the present invention may be utilised in a monitoring system as described in the Applicant's earlier international patent publication WO2018/068087 and whereby a plurality of the sensor apparatus may be distributed in a two-dimensional or three-dimensional spatial arrangement within a porous material such as an ore heap to acquire data from various regions within a bulk heap.

The present invention may also be applied to smaller scale leach applications such as tank and crib leaching, as well as large scale dump leaching. The present invention provides continuous spatial monitoring over time of leaching heaps and their chemical parameters. This, in turn, allows for real-time control of parameters in the heap leach, for example temperature, chemical parameters, chemical concentrations (acid, for example), air flow, liquid flow, aeration, spacing for irrigation and the like. Advantageously, real-time control of these parameters allows for improved leaching recovery and efficiency, the possibility to take remedial action quickly in response to changes in the heap, as well as a reduction in consumables (i.e. the materials used in leaching).

The invention is particularly advantageous in that it may be implemented for use in partially saturated porous media environment with non-steady state and slow flow. For example, an environment where liquid is gradually seeping through the media such as damp particulates, for example an irrigated leaching solution moving slowly downward through an ore heap. The invention may advantageously create a locally saturated condition within the apparatus sensor housing to facilitate measurement by sensors otherwise incapable of operation in a partially saturated environment due to an insufficient contact interface between the liquid and the sensor(s). In this regard, the apparatus may be configured to a flow harvesting operation whereby a limited amount of received flow is collected, choked/dammed and directed in a particular direction in order to enable or facilitate sensor measurement.

In this regard, an embodiment of the invention provides an apparatus configured for location within a partially saturated porous media region and for forming a locally saturated condition for at least one sensor of the sensor arrangement to facilitate data acquisition by the sensor arrangement. The apparatus may therefore operate to collect liquid from a relatively slow flowing liquid passing through the porous media and choke/dam a limited amount of liquid at the sensor arrangement in order to enable data acquisition from the liquid.

According to an embodiment of the invention, the flow between the inlet and the outlet is configured as a gravity feed wherein the inlet is located at a higher level than the outlet and the sensor arrangement located at a level below the inlet and above the outlet. For example, the inlet may be positioned at an upper side of the sensor housing and orientated so as to face upwardly within the region of porous media. In a form of the invention the inlet extends along a substantial portion of an upper side of the sensor housing and wherein, in use, the inlet is upwardly facing. In a particular form of the invention, the inlet has a planar configuration and is, in use, approximately horizontal.

In alternative configurations, liquid flow may be promoted by means other than gravity feed such as capillary (i.e. ‘wicking’) flow or via a pump. In this regard, it is envisaged that the inlet need not necessarily be located above the sensor arrangement and could be located elsewhere relative to the sensor arrangement with an appropriate configuration that allows liquid to be received or drawn through the inlet and toward the sensor arrangement.

In an embodiment of the invention, an interior of the sensor housing provides a sensor chamber in which the sensor arrangement is housed. In an embodiment, the inlet may be located above the sensor chamber and, in particular, vertically above the sensor chamber. The inlet may be configured in size or shape or formation so as to maximise the amount of liquid received by the inlet and delivered to the sensor chamber. In an embodiment, the inlet is configured to occupy all or most or a substantial portion of an upper side of the sensor housing. In an embodiment, the inlet is configured with an area approximately equal to two-dimensional footprint of the sensor chamber. The inlet may therefore be sized to span the entire length and width of the sensor chamber.

In an alternative embodiment which may be desirable in particularly porous media applications with particularly low liquid flow rates, the apparatus may be provided with an area larger than the footprint of the sensor chamber. Alternatively, the apparatus may include a collection arrangement having a larger area than the inlet and which captures and directs collected liquid toward the inlet. For example, the apparatus may include one or more collection members extending outwardly (and potentially upwardly) from the inlet which provide a larger collection area than the area of the inlet and which are inclined relative to horizontal so as to direct collected liquid toward the inlet.

In an embodiment of the invention, in circumstances where the fluid flow in the system is very low or abnormally low and which may contribute to the formation of unfavourable saturation conditions in the sensing chamber, then the inlet is configured to have a funnel or sloping shape and extend to an area wider than the housing to capture and accumulate more liquid and maintain the correct locally saturated conditions in the sensor chamber.

As well as the sensor chamber below the inlet which houses the sensor arrangement, the apparatus may include one or more additional chambers which house electrical componentry such as printed circuit boards (PCBs) or other electrical componentry configured to receive electrical signals from the sensors and/or to transmit the sensor data to a connected receiving hub via a wired or wireless connection. The sensor chamber may be fluidly isolated (i.e. fluidly sealed) from the electrical componentry chamber in order to prevent sensitive electrical componentry exposure to leaching solution or potentially corrosive liquids such as lixiviant in the porous media and in the sensor chamber.

The sensor arrangement within the sensor chamber may be connected to corresponding electrical componentry within the electrical componentry chamber via data cables extending through a wall or partition between the sensor chamber and electrical componentry chamber. In an alternative configuration, the one or more sensors may extend through a wall or partition between the sensor chamber and electrical componentry chamber such that a sensing part of the sensor is located within the sensor chamber for exposure to the received liquid and a non-sensing part or other part of the sensor is located within the electrical componentry chamber.

The electrical componentry chamber may include electronics configured to convert analogue signals received from the one or more sensors into a digital signal. In embodiments of the invention where the sensor arrangement includes a dissolved metal ion sensor, the chamber may include a local PCB with electronics configured to perform electrochemical or optical measurements required for the dissolved metal ion and dissolved oxygen sensing.

The outlet may be located at the base of the sensor housing and, for example, at the base of the above-noted sensor chamber such that liquid flows generally downward through the sensor chamber and through the outlet. In a form of the invention, the outlet is configured to promote a relatively small volume of liquid to pool at the outlet. In a particular embodiment, the outlet has a smaller area than the inlet to delay fluid egress from the sensor housing. The relatively smaller outlet area may restrict or limit or choke/dam egressing fluid flow and cause a pooling of liquid upstream of the outlet. This configuration could facilitate the operation of certain sensor types which require or are optimised for exposure to a larger quantity of fluid or a more saturated condition. Accordingly, this configuration may be desirable in some fluid monitoring applications and unnecessary in others. The ratio of the outlet area to inlet area may also vary depending on application and depending on the amount of fluid pooling that is required (if any).

In a particular form of the invention, the sensor arrangement includes a conductivity sensor which is located at a base of the sensor housing. The conductivity sensor may be positioned so as to be exposed to a pool of liquid at the housing base. In use, the conductivity sensor may be partly surrounded by or potentially submerged within a pool of liquid at the outlet.

The configuration of the sensor housing may be such that the sensor chamber within the sensor housing is sealed from the porous material other than the inlet and the outlet. For example, the sensor housing may have no exterior openings other than the inlet and the outlet. In this manner, the potentially corrosive and aggressive liquids and chemicals present in the porous material are permitted passage, in use, through the housing only via the inlet and only via the predetermined flow path. The apparatus may therefore be configured to manage or control exposure of the sensor arrangement to the (potentially aggressive) liquids intended for analysis. The flow path which is permitted through the sensor housing may be configured/designed or tailored for particular sensors to make direct contact with the received liquid and for other sensors to be maintained out of direct contact.

In this regard, the sensor arrangement may include one or more sensors located out of contact with the flow path. For example, sensors which can operate in fully unsaturated conditions. The sensor arrangement may include a temperature sensor spaced apart from the flow path and thereby configured to measure internal temperature within the sensor chamber which is akin to the temperature of the liquid within the porous media and therefore also the temperature of the porous media (for example the temperature of a heap) In an alternative embodiment, the temperature sensor may be placed in direct contact with the flow of the liquid, or near direct contact with the liquid.

In a particular embodiment of the invention, the apparatus is provided with a directing arrangement configured to direct liquid received through the inlet toward the sensor arrangement. The directing arrangement may be configured to channel or funnel received liquid toward the sensor arrangement. The directing arrangement may be configured to funnel received liquid at a particular location such as a particular location on the sensor arrangement. The predetermined flow path provided by the apparatus may be managed, at least in part, by the directing arrangement which could help direct or promote flow through the sensor housing along the preferred flow path.

In an embodiment of the invention, the directing arrangement is located between the inlet and the sensor arrangement and is configured to funnel liquid collected from the inlet toward the sensor arrangement. For example, the directing arrangement may operate to funnel received liquid collected across a larger volume or area through a relatively small gap or passageway in order to direct the received liquid toward the sensor arrangement and in order to maximise the volume of liquid at the sensor arrangement available for measurement. The directing arrangement may thereby operate to increase or maintain the saturated conditions at the sensing site and enable sensors to operate correctly.

The directing arrangement could have a variety of configurations. In a particular embodiment of the invention, the directing arrangement includes at least one inclined surface extending below the inlet and terminating at a lower edge orientated toward the sensor arrangement and wherein, in use, the liquid flow is collected or formed on the inclined surface and flows toward the lower edge and from the lower edge toward the sensor arrangement. The inclined surface could be curved or could form part of a conical-shaped structure. Alternatively, in a particular form of the invention the inclined surface is generally planar.

The inclined surface may therefore provide a surface below the inlet at which liquid received through the inlet is collected and subsequently flows downward under the influence of gravity toward the lower edge of the inclined surface whereupon it could immediately contact the sensor arrangement or, alternatively, could fall (e.g. drip or stream) from the lower edge onto the sensor arrangement.

In a particular form of the invention, the directing arrangement includes a pair of inclined surfaces. For example, a first inclined surface may have a lower edge terminating above a second and lower inclined surface and whereby liquid collected on the first inclined surface is directed to the second inclined surface before being directed toward the sensor arrangement. The first and second inclined surfaces may extend beneath separate portions of the inlet such that all of the inlet is above one or the two inclined surfaces. This configuration may advantageously allow for received fluid to be collected on one or both of the inclined surfaces across an entire width of the inlet and be directed onto the sensor arrangement at various positions within the sensor housing depending on the length and position of the lower of the two inclined surfaces.

In a particular form of the invention, the sensor housing includes one or more internal ledges for supporting the fluid directing arrangement. For example, the sensor housing may be provided with a pair of internal ledges at an upper portion of the sensor chamber which are configured to each support one of the pair of inclined surfaces of the directing arrangement. The ledges may be provided at differing levels within the sensor chamber so as to correspond with different levels of the inclined surfaces. The sensor housing may be generally elongate. The internal ledges and inclined surfaces of the fluid directing arrangement may therefore also be generally elongate.

As noted, one or more sensors of the sensor arrangement are located at the flow path for contacting the liquid in order to obtain sensor data from the liquid. Depending on the number and type of sensors provided in the sensor housing (which may depend on the particular liquid monitoring application) the predetermined flow path may be configured so as to flow across or past the one or more sensors in a sufficiently high volume or stream to ensure accurate sensor performance. However, according to a particular embodiment of the invention, the apparatus may include moistening material in contact with the one or more sensors of the sensor arrangement, the moistening material positioned within the flow path and configured to maintain a moist or saturated condition at the one or more sensors to facilitate measurement of one or more properties of the liquid by the one or more sensors.

The moistening material may advantageously ensure a saturated condition is maintained for the sensors in instances where flow rate is insufficiently low or inconsistent to provide the necessary sensor saturation for accurate sensor operation. The moistening material may therefore not be required in all applications such as applications where flow rate within the porous media is sufficiently high. As well as facilitating a saturated condition in low flow rates, the moistening material may advantageously enlarge the saturated area available for measurement and thereby enable the use of additional: sensors and/or an optimised physical arrangement/location of sensors.

For example, in a particular embodiment of the invention the sensor arrangement includes a plurality of sensors in contact with the moistening material and wherein at least one sensor contacts a first side of the moistening material and at least one sensor contacts a second and opposite side of the moistening material. The first and second sides of the moistening material may be, for example, an upper and a lower side. The moistening material may therefore facilitate the location of sensors on various sides of the moistening material and thereby providing an enlarged saturated surface area available for sensor measurement. The moistening material may be positioned in a gap or space between sensors on opposite sides of the flow path. The moistening material may be generally elongate in shape and, in particular, could have a generally planar elongated shape.

The moistening material may have a material configuration which allows for liquid to be distributed via capillary motion throughout the moistening material but whilst also allowing for flow to continue through the moistening material and for flow to exit a lower side of the moistening material and continue along the flow path toward the outlet.

It should be appreciated that references herein to liquid ‘flow’ are therefore intended to encompass relatively slow-moving and non-steady-state flow such as diffusion or capillary flow or wicking.

The moistening material may have a further advantage in that it may be configured as part of the filtration arrangement to remove relatively fine particulates (solids such as fines and clays) from the liquid. This aspect will be discussed subsequently in further detail when the filtration arrangement is described in further detail.

The sensor arrangement may include one or more of a variety of sensors depending on the particular liquid monitoring application for which the sensor apparatus is being used. The sensor arrangement may include one or more of a pH sensor, a dissolved metal ion sensor, a temperature sensor, a redox potential sensor, a dissolved oxygen sensor or a conductivity sensor. In a particular form of the invention the sensor arrangement includes all six of these sensor types. In a particular form of the invention, one or more of the six sensor types may include multiple discrete sensors. For example, multiple metal ion sensors may be provided for detecting various species of metal ions. It will therefore be appreciated that the sensor arrangement may include more than six sensors.

It will be appreciated that the sensors may take any suitable form depending on the physical and/or chemical phenomena to be measured, for example ion-selective electrodes, voltammetry electrodes, amperometric sensors and optical sensors, such as dissolved metal ion, dissolved oxygen and sulphide sensors.

The sensor arrangement may include a reference electrode and wherein one or more sensors of the sensor arrangement are associated with the reference electrode. The present invention may utilise any suitable reference electrode having adequate chemical stability, acid resistance, thermal stability and mechanical stability. In a particular form of the invention, the sensor arrangement includes a solid-state reference electrode which provides improved longevity, robustness and resistance to chemicals and high temperatures and mechanical impacts. The solid-state reference electrode may take any form and may be, for example, like the type described in the Applicant's earlier international patent publication WO2018/201200.

The reference electrode may be utilised in combination with the one or more sensors for electrochemical measurements. In a form of the invention, the reference electrode is associated with a pH sensor in the sensor arrangement for measuring pH levels of the received liquid.

In an embodiment of the present invention, the sensor arrangement includes a solid-state pH sensor. Typical pH sensors are ion-selective glass electrodes which often have an internal reference electrode. Glass pH electrodes have high impedance and require special electronic devices to log the signal-they also suffer from alkali error in high pH environments and acidic error when the pH is very low. They are also fragile and require constant calibration due to drift and instability. Further, some solid-state sensors such as ion-sensitive field-effect transistor ISFET sensors also suffer from drift and hysteresis effects and are sensitive to light. The use of a solid-state pH sensor may therefore be particularly desirable due to the harsh operating environment of the present invention. The solid-state pH sensor may take any form and may be for example like the type described in the Applicant's earlier international patent publication WO2016/033632 for a Metal Oxide pH sensor.

In a particular form of the invention, the sensor arrangement includes a pH sensor configured for measuring the pH of the received liquid and the pH sensor is positioned along a lower side of the flow path. The pH sensor may include a sensing surface having an inclined orientation configured for the liquid flow to move over the sensing surface. In a form of the invention, the direction of the flow path over the sensing surface is diagonally downward through the sensor housing. In a particular embodiment, the sensing surface is orientated at approximately 30° to horizontal (and also 30° relative to the planar inlet of the sensor housing). It will be appreciated that various inclinations/orientations are possible. The inclination of the pH sensor sensing surface promotes fluid flow down and across the sensing surface so as to reduce or eliminate flow stagnation which may cause precipitant to form and/or which may begin to compromise data accuracy in that the sensors are no longer exposed to freshly received liquid from within the porous region. Various configurations or geometries of the pH sensor and the pH sensor sensing surface are envisaged. In an embodiment, the sensing surface of the pH sensor is curved and, for example, rounded. In a particular embodiment, the pH sensor has a cylindrical configuration. In a particular embodiment, the pH sensor sensing surface is cylindrical or is configured as a segment of a cylinder. In an alternative embodiment, the sensing surface of the pH sensor is generally planar.

In an embodiment of the invention, the sensor arrangement includes one or more additional sensors spaced from the sensing surface of the pH sensor by a gap and the flow path extends through the gap between the sensing surface and the one or more additional sensors. In a particular embodiment of the invention, a solid-state reference electrode and a dissolved metal ion sensor are positioned along an upper side of the flow path. The solid-state reference electrode and dissolved metal ion sensor may be positioned on the opposite side of the flow path from the sensing surface of the pH sensor. The sensor arrangement may further include a redox sensor positioned along an upper side of the flow path.

In an embodiment of the invention, the moistening material may be located on the sensing surface of the pH sensor. The moistening material may be configured in shape or size so as to extend along most or substantially all of the sensing surface. The moistening material may therefore be inclined at the same inclination as the sensing surface and, in particular, may be, in use, inclined at approximately 30° relative to horizontal. Received liquid may thereby flow diagonally downwards through the moistening material. In this regard, an upper end of the moistening material may be an ‘upstream’ side of the moistening material and a lower end of the moistening material may a ‘downstream’ side of the moistening material. The upstream end of the moistening material may be located at or may overlie an upper part of the sensing surface and the downstream end of the moistening material may be located at or may overlie a lower part of the sensing surface.

In an embodiment of the invention, the upstream end of the moistening material is positioned below (for example, directly vertically below) the directing arrangement such that the directing arrangement funnels flow onto the upstream end of the moistening material. For example, the lower edge of the inclined surface of the directing arrangement may be positioned vertically above the upstream end of the moistening material such that received liquid will drip or stream from the directing arrangement onto the moistening material.

The received liquid can thereafter diffuse through the moistening material and supply/create a saturated condition for all sensors in contact with the moistening material so as to facilitate measurement of liquid properties by the sensors.

The moistening material may act to filter fine particulates by allowing diffusion of liquid but not solid particulates. In this manner, fluid may be passed through the moistening material from the upstream (i.e. upper) end of the moistening material to the downstream (i.e. lower) end of the moistening material whilst particulates are captured by the moistening material and not generally diffused toward the outer surfaces of the moistening material where the sensors are located. The moistening material may thereby operate to prevent or reduce sensor exposure to fine particulates which have passed through the first and second filters. The sponge filter are the combination of hydrophobic fibres and hydrophilic fibres. The mass ratio has been adjusted to allow the liquid reaching the sensors' surface for sensing, but also not blocking the liquid too long and missing the change from the bulk. The filter materials can be the following materials by themselves or mixed: nylon, polypropylene, polycarbonate, polyester, viscose cellulose esters, PMMA, Teflon, HDPE, glass fibre, quartz fibre, and graphitic polymer mixed filter or membrane materials.

The downstream end of the moistening material may be located at a higher level than the outlet. In a particular form of the invention, the outlet may be located at a base of the sensor housing. The downstream end of the moistening material may be located above the base such that fluid exiting the downstream end of the moistening material will stream or drip under influence of gravity from the downstream end and toward the base.

The invention is advantageously provided with a filtering arrangement to prevent or reduce ingress of particulates from the porous media into the sensor housing which may, over time, foul or damage sensors or obstruct the flow path of liquid through the sensor housing. The filtering arrangement is advantageously configured to remove particulates (i.e. remove at least some particulates) from the liquid prior to the liquid contacting the one or more sensors. It will be appreciated that the function of the filtering arrangement is to reduce or minimise the amount of particulates to which the sensors are exposed. Accordingly, it will be appreciated that the filtering arrangement need not necessarily removal all particulates and that a certain level of relatively small particulates may still remain which has passed through the filtering arrangement. In this instance, the filtering arrangement has operated to reduce the amount of particulates exposure for the sensors, as compared to the particulates exposure for the sensors if the filtering arrangement were not present.

The filtering arrangement could be provided in a variety of configurations. In an embodiment of the invention, the filtration arrangement includes a first stage filter located at the inlet and configured to prevent ingress of relatively large particulates from the porous media. In a particular embodiment, the first stage filter includes a rigid mesh located at the inlet, the rigid mesh including openings sized to prevent ingress of particulates sized larger than approximately 2 mm in width or diameter. In a form of the invention, the rigid mesh includes a plurality of inlet openings which allow only liquid and particulates of sufficiently small size to be received into the sensor housing. The inlet openings may be sized to as to prevent ingress of relatively large size particulates within the porous media.

The rigid mesh may therefore act as a primary filter for large-size particulates but allowing liquid and smaller particulates to ingress the sensor housing. In an embodiment of the invention, the rigid mesh is integrally formed with the sensor housing. The inlet openings of the rigid mesh may have a variety of shapes or configurations and, for example, may be circular or may be square or triangular or diamond shaped. The inlet openings may have an average size of approximately 1 to 2 mm in width or in diameter. In an embodiment of the invention, the inlet openings have an average size of approximately 2 mm length×2 mm in width or, alternatively, approximately 2 mm in diameter.

The rigid mesh may have a generally planar configuration and wherein the plane of the rigid mesh is, in use, approximately horizontal in order to capture downwardly moving liquid within the porous media. The inlet openings of the rigid mesh provide passageways that are approximately vertical or otherwise generally perpendicular to the plane of the rigid mesh.

As noted in the foregoing, the inlet may be provided with an area that spans the whole footprint of the sensor chamber within the sensor housing. The rigid mesh may therefore be sized to span the width and/or length of the sensor chamber. The rigid mesh may extend all or substantially all of the way to one or more sidewalls of the sensor housing. The rigid mesh may cover the entire or substantially entire top side of the sensor housing.

In an embodiment of the invention, the filtration arrangement includes a multi-stage configuration for removing particulates of varying sizes. In a particular embodiment, the filtration arrangement includes a second stage filter configured to filter material which has passed through the first stage filter and to remove smaller-sized particulates than the first stage filter. The second stage filter may be located below the first stage filter. In an embodiment of the invention, the second stage filter is located vertically below the first stage filter. The second stage filter may have an elongate configuration and may extend generally horizontally along an underside of the first stage filter. The second stage filter may be sized so as to extend across all or substantially all of an underside of the inlet. In this manner, all of the material passed through the first stage filter may be received by the second stage filter.

The second stage filter may be a sponge or sponge-like filter material. The second stage filter may include a nylon sponge filter material. The second stage filter may have a resilient configuration which allows resilient compression of the second stage filter and whereby the second stage filter is resiliently compressed into a volume of the sensor chamber below the inlet. The second stage filter may be configured in size or shape or resiliency or other parameter for insertion into an upper portion of the sensor chamber within the sensor housing. The second stage filter may be configured to remove particulates sized in the range of 10 micrometre to 2 mm.

The filter materials can be the following materials by themselves or mixed: nylon, polypropylene, polycarbonate, polyester, viscose cellulose esters, PMMA, Teflon, HDPE, glass fibre, quartz fibre, and graphitic polymer mixed filter or membrane materials.

The second stage filter may include one or more of the following materials including nylon, polypropylene, polycarbonate, polyester, viscose cellulose esters, PMMA, Teflon, HDPE, glass fibre, quartz fibre, and graphitic polymer mixed filter or membrane materials. The material(s) selected for use in the second stage filter may be selected for tolerance to the particular properties of the leaching solution of lixiviant used in the intended application of the sensor apparatus.

In an embodiment of the invention, the filtration arrangement includes a third stage filter for filtering liquid which has passed through the second stage filter and to remove smaller-sized particulates than the second stage filter. The third stage filter may be configured to remove particulates sized in the range of <1 micrometre to 100 micrometres.

In an embodiment of the invention, the third stage filter is provided with anti-fouling properties to reduce or resist biofouling at the sensor arrangement. The third stage filter may be provided with anti-fouling fibres selected for bactericidal and/or fungicidal and/or algicidal properties. For example, the third stage filter may include silver-based fibres or silver-based compounds to provide an inhibitory effect against microorganism growth at or in the vicinity of the sensor arrangement. The third stage filter may include a composite of different materials selected to provide the filter with specific filtering and/or moistening and/or liquid diffusion and/or antifouling properties. The third stage filter may include a nylon or wool sponge-type structure incorporated with anti-fouling fibres.

The third stage filter may include a combination of hydrophobic fibres and hydrophilic fibres. In a particular embodiment, the mass ratio may be configured to allow liquid to reach the sensors' surface for sensing, but also not blocking or restricting flow the liquid through the filter. The filter materials might include one or more of the following materials: nylon, polypropylene, polycarbonate, polyester, viscose cellulose esters, PMMA, Teflon, HDPE, glass fibre, quartz fibre, and graphitic polymer mixed filter or membrane materials.

In an embodiment of the invention, the second and/or third stage filter may have chemical or physical properties which enhance selective capture or adsorption of particular particulates. For example, the second and/or third stage filter may include a positively or negatively charged material configured to electrostatically adsorb negatively or positively charged particulates. The second and/or third stage filter may be provided with hydrophobic properties in order to attract hydrophobic particles (or vice versa). In another example, the second and/or third stage filter may be provided with a specific chemical coating allowing chemisorption of chelating of particular particulates of certain surface chemistry. In another example, the second and/or third stage filter may be coated with a specific surfactant or flocculant which aids or promotes the aggregation of ultrafine particulates into larger agglomerates of sufficient size to be captured by the second and/or third stage filter. The second and/or third stage filter may have a particular morphology such as a sponge particle shape, porosity or surface area that promotes entrapment or capture or attraction of particular particulates of specific shape or size or molecular structure.

In a particular embodiment, one or more of the above-noted filter features are provided in the second stage filter and one or more different filter features of the above-noted features are provided in the third stage filter. For example, some of the above-noted filter features may be more compatible with the second stage filter which is configured for capturing less-fine particulates than the third stage filter which is more prone to becoming blocked. In a particular non-limiting example, the second stage filter (and not the third stage filter) may be coated with a flocculant which aids or promotes the aggregation of particulates.

As noted in the foregoing, a moistening material may be used to promote a saturated condition at one or more of the sensors and in a particular form of the invention, the moistening material may be configured as part of the filtration arrangement. In a particular embodiment, the third stage filter of the filtering arrangement is provided by the moistening material. The moistening material may therefore be configured to act as both a filter for relatively fine particulates as well as a wetting or moistening material which diffuses liquid (for example via capillary flow) in order to promote a wet or saturated condition for one or more of the sensors. For example, to wet the surface of the sensors in contact with the moistening material. The moistening material may be configured with a third function of inhibiting biofouling insofar as it may be provided with anti-fouling properties of the type mentioned above.

The physical or material characteristics of the moistening material may be selected with a desired level of diffusion to promote or maintain a desired level of wetness or saturation to facilitate sensor operation whilst also having a desired level of permeability allowing for flow to pass through the moistening material and continue on the flow path toward the outlet.

In an embodiment of the invention, the outlet may be provided by a rigid mesh formation. The outlet mesh formation may include a plurality of outlet openings which allow liquid to egress the sensor housing whilst preventing relatively large particulate from entering via the outlet, for example during installation of the sensor apparatus into the porous media.

It will be appreciated from the above discussion of the filtration arrangement that a sensor apparatus according to the invention may be configured to be submerged or buried within the porous media. The region of porous media in which the sensor apparatus is located may be below a surface of the porous media. According to an embodiment of the invention, the entire sensor apparatus may be configured to be buried or submerged within the porous media.

The sensor housing may be formed of a variety of materials and the selected material may vary depending on the intended application of the sensor apparatus and particularly whether the apparatus is intended for use in an aggressive or corrosive environment. In a particular form of the invention, the housing includes a corrosion-resistant polymer material. In a particular embodiment, the sensor housing is formed of a polycarbonate material. In a particular form of the invention, the sensor housing includes a metallic material. For example, a corrosion-resistant metallic material. In a particular form of the invention, the sensor housing includes a titanium material and, for example a titanium alloy material. In a particular form of the invention, the housing is formed via additive manufacturing. The use of additive manufacturing may facilitate tailor made production of the rigid mesh formation of the housing inlet and the housing outlet and may allow for the inlet and outlet to be integrally formed with the sensor housing. In a particular embodiment, the sensor housing is produced via additive manufacturing of a metallic material.

In a particular embodiment, the sensor housing includes a strengthening or reinforcement arrangement. More particularly, the sensor housing may include strengthening ribs to strengthen the housing against relatively high loading when used in a tall ore column. In a particular embodiment, the strengthening ribs are formed of stainless steel. The strengthening ribs may be completely encapsulated in the polycarbonate material of the sensor housing. In a particular embodiment, the sensor housing is additively manufactured (for example, 3D printed) with voids configured for the strengthening ribs to be subsequently inserted and then resin sealed.

In a particular form of the invention, the sensor housing is selectively openable to enable access to the sensor chamber. This configuration may advantageously allow access to the sensor chamber for inspection, cleaning, maintenance, repair or replacement of sensors or other internal components such as the second stage filter or the moistening material. For example, if the second stage filter and the moistening material become blocked with filtered particulates over time requiring cleaning or replacement.

The sensor housing may be configured with a removable part such as a removable lid or end. In a particular embodiment, the sensor housing may include a removable end. In one form of the invention, the removable end includes the fluid directing arrangement such that removal of the removable end also removes the fluid directing arrangement from the sensor housing. For example, the one or two inclined surfaces of the fluid directing arrangement may extend from the removable end and, in particular, may be integrally formed with the removable end.

Accordingly, in an embodiment of the invention, the directing arrangement is removable from the sensor housing. In an alternative embodiment, the directing arrangement may be integrated with the sensor housing. For example, the directing arrangement may be non-removably integrated with the sensor housing. In particular, the directing arrangement could be integrally formed with the sensor housing and the inclined surfaces could be integrally formed with walls of the sensor housing.

The apparatus may be provided with an appropriate sealing configuration at the interface of the removable end and the rest of the sensor housing. The engagement between the removable end and the rest of the sensor housing may be provided by an interference fit, snap fit, latching mechanism, clamping mechanism or any other mechanism or configuration suitable to allow for selective disconnection and then reconnection between the removable end and the rest of the housing.

It will be appreciated from the foregoing that embodiments of the present invention advantageously provide a sensor apparatus configured to receive relatively limited flows of liquid in porous media and to direct the fluid received along a managed flow path intended to maximise fluid availability for sensor measurement. In certain embodiments, the predetermined flow path may include:
a. ingress through the sensor housing inlet which also operates as a first stage filter to prevent ingress of relatively large particulates;
b. passage through the second stage filter configured to filter medium-sized particulates from the liquid;
c. passage through the flow directing arrangement configured to funnel liquid exiting the second stage filter toward the sensor arrangement and in particular, toward an upstream end of the moistening material located on the sensing surface of the pH sensor;
d. diffusion of the liquid through the moistening material to achieve a more saturated condition for the one or more sensors arranged in contact with the moistening material;
e. passage through the downstream end of the moistening material and subsequent choking/damming of liquid at the base of the housing adjacent to the outlet;
f. egress through the sensor housing outlet and return to the porous media and, in particular, the portion of porous media below the sensor housing.

It will be appreciated from the above that the predetermined fluid flow path allows for the sensor arrangement to be configured according to the particular requirement/application of certain sensors. For example, certain sensors requiring wet or saturated operating conditions may be positioned in contact with the moistening material or at the base of the housing. Sensors may be positioned on opposite sides of the moistening material in order to utilise the saturated surface area of the moistening material. Other sensors which do not require direct contact with the liquid may be positioned at other places within the sensor housing.

It will also be appreciated from the above discussion that embodiments of the invention are configured to maintain a flow of liquid through the sensor housing (for example, via inclination of the pH sensor and moistening material) so as to prevent or minimise the occurrence of stagnant zones which could promote fouling and result in measurement of liquid samples that are have not been recently collected from the porous media and therefore may not be representative of the true characteristics of the liquid flowing through the porous media at the time.

Embodiments of the invention therefore provide for near real-time data acquisition of chemical and physical properties of liquid samples that are received in-situ and thereby significantly more representative of the true conditions within the porous media as compared to ex-situ liquid samples such as samples collected at the base of a column or a heap, in which liquid properties may have changed since exiting the porous media.

Furthermore, in some applications, liquid properties may change since exiting the region of the porous media desired for analysis. For example, in some applications it may be desirable to acquire liquid data from a relatively shallow region of an ore heap and before the liquid has passed through the entire depth of the heap and is collected at the base. The present invention may allow for data acquisition at various depths of porous material and enable comparison of liquid properties across the changing spatial aspects of the bulk porous media.

In this regard, another aspect of the present invention provides for a system for acquiring data from liquid in a heap leaching application including a plurality of the above-discussed sensor apparatuses and wherein the sensor apparatuses are distributed throughout regions of heap leaching material to be analysed and the sensor apparatus configured to acquire and transmit sensor data about the liquid within the region of each sensor apparatus to one or more hubs. For example, a number of sensor apparatuses according to the present invention may be located at different regions corresponding to different depths of the heaped material in order to acquire in-situ data from the discrete regions.

Another aspect of the invention may provide a column leaching apparatus including an upright column having a peripheral wall which includes an opening and the column leaching apparatus further including a sensor apparatus as discussed in the foregoing and wherein the sensor apparatus extends through the opening in the peripheral wall and into an interior of the column. The opening of the upright column may be configured to receive the sensor housing and to provide a seal between an edge of the opening and the sensor housing. In a particular embodiment, part of the sensor apparatus such as the sensor housing may be located within the column and part of the sensor apparatus such as the electrical componentry chamber may be located outside of the column and connected via one or more data cables to a receiving hub configured to receive data from the sensor housing about liquid within

In some columns in laboratory settings an external water jacket lines/surrounds the column and is used to facilitate the transport of hot or cold fluid (mostly water is used) around the column to maintain a particular temperature within the column. In this embodiment, the sensor apparatus may be configured so as to extend through the jacket lining and into the column as described above without any interference or disturbance of the jacketed heating system or fluid flow.

A further aspect of the present invention may provide for a method of acquiring data from liquid within a porous media to be analysed including the steps of locating one or more of the above-discussed sensor apparatuses within the porous media, the one or more sensor apparatuses being configured periodically acquire sensor data relating to chemical or physical properties of the fluid surrounding the sensor over a predetermined period of time and to periodically transmit the sensor data via the a data link to one or more hubs. For example, the sensor data may be collected and transmitted in a manner that is described in the Applicant's earlier international patent publication WO2018/068087.

### BRIEF DESCRIPTION OF DRAWINGS

The present invention will now be described with reference to the figures of the accompanying drawings, which illustrate preferred embodiments of the present invention, wherein:

FIG. 1 is a perspective view of a sensor apparatus according to an embodiment of the invention and a column leaching apparatus;

FIG. 2 is a perspective view of the sensor apparatus and column leaching apparatus of FIG. 1 following insertion of the sensor apparatus into the column leaching apparatus;

FIG. 3 is a perspective view of a sensor housing of the sensor apparatus of FIGS. 1 and 2;

FIG. 4 is an underside view of the sensor housing of the previous figures and illustrates a base of the sensor housing;

FIG. 5 is an end view of the sensor housing of the previous figures with an end cap removed;

FIG. 6 is an exploded view of sensor housing of previous figures illustrating insertion of the end cap into the sensor housing;

FIG. 7 is a sectional end view of the sensor apparatus of the previous figures located within a porous media;

FIG. 8 illustrates sensor apparatus of FIG. 7 in use with a liquid within the porous media flowing through the sensor apparatus;

FIG. 9 illustrates the sensor apparatus of the previous figures in use within a column leaching apparatus; and

FIG. 10 illustrates a plurality of the sensor apparatus of previous figures distributed throughout a heap in a heap leaching process.

### DETAILED DESCRIPTION

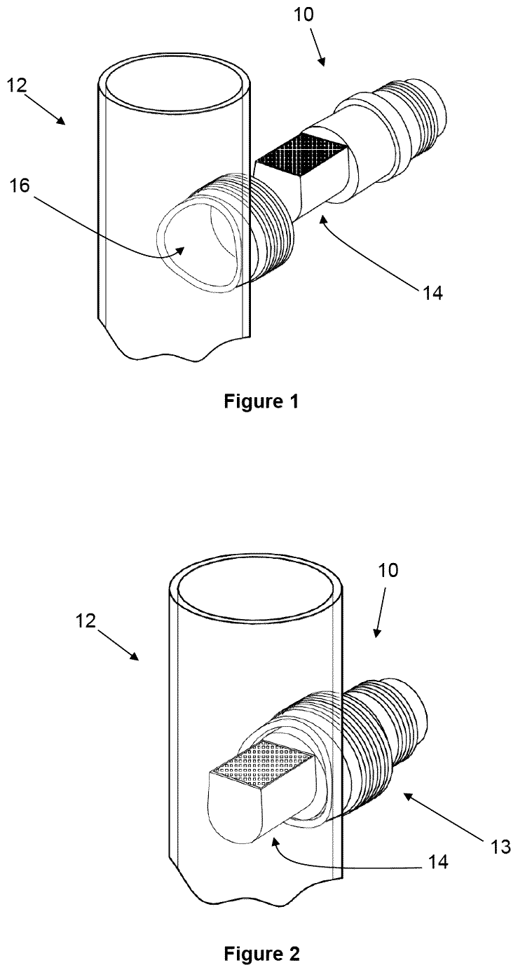

FIGS. 1 and 2 illustrates a sensor apparatus 10 and a column 12 of a column leaching apparatus. The sensor apparatus 10 includes a sensor housing 14 configured for insertion through an opening 16 in the column 12. FIG. 1 illustrates the sensor apparatus 10 removed from the column 12.

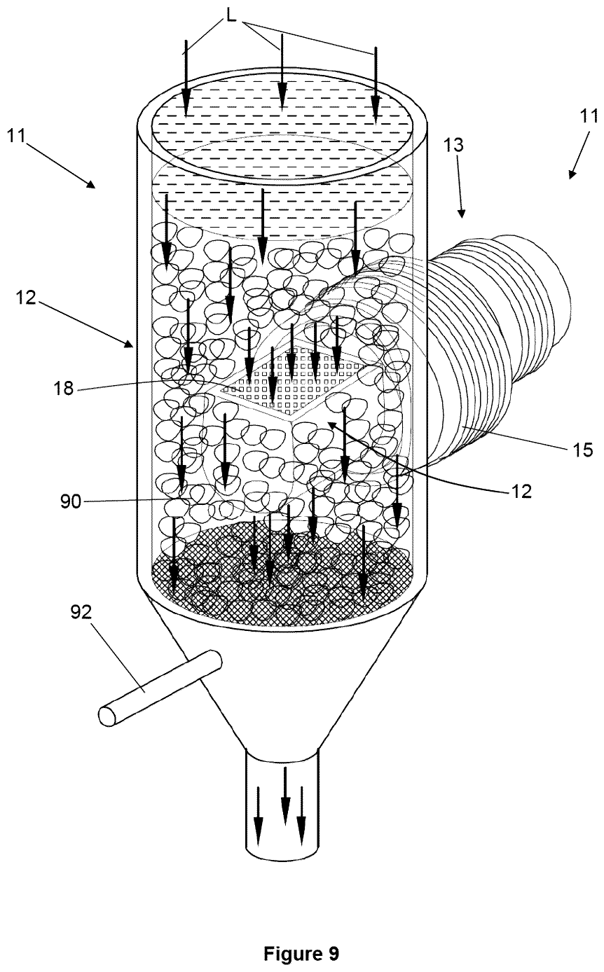

FIG. 2 illustrates the sensor apparatus inserted into the column 12 such that the sensor housing 14 is located within an interior of the column 12. A portion 13 of the sensor apparatus 10 remains outside of the column 12 and which will be discussed in further detail below when FIG. 9 is subsequently described.

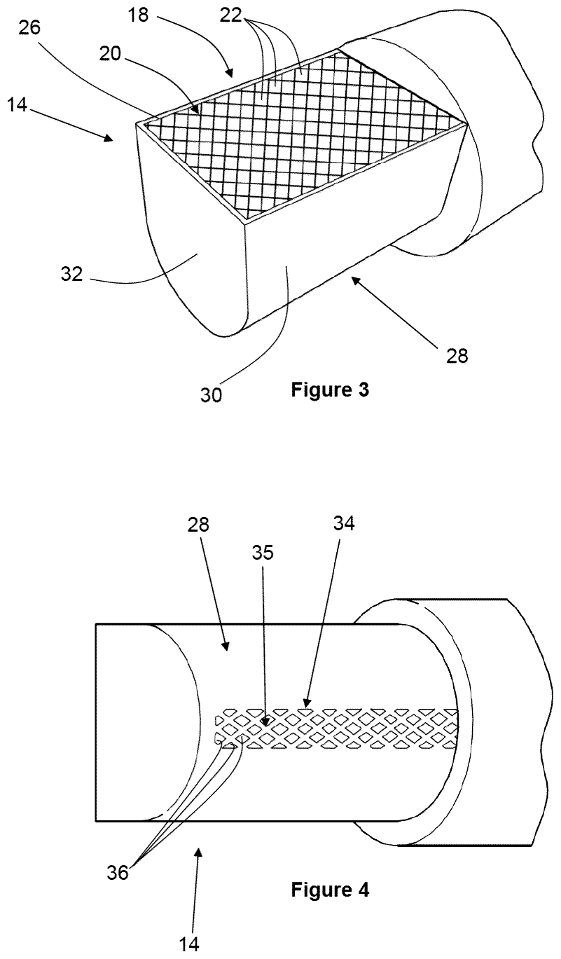

FIGS. 3 to 6 provide various closer perspectives of the sensor housing 14.

With reference to FIG. 3, the sensor housing 14 includes an upwardly facing inlet 18 occupied by a rigid mesh 20. The rigid mesh 20 has a generally planar configuration and includes a plurality of openings 22 allowing liquid to be received through the rigid mesh 20 into the sensor housing. The openings 22 are sized to prevent ingress of relatively large solids such as stones, rocks or large clumps or dirt, ore, clay or the like. The rigid mesh 20 is thereby configured as a first stage filter to prevent or limit ingress of relatively large particulates from porous media in which the sensor housing is located during use.

The sensor housing 14 and the inlet 18 may be typically use in the orientation shown in the illustrated embodiments and wherein the inlet is orientated generally horizontally. In this context, the inlet 18 may therefore be described as upwardly orientated or upwardly facing. With the inlet 18 in a horizontal orientation, the area of the inlet 18 perpendicular to the force of gravity is maximised so as to maximise the amount of liquid collectable through the inlet 18 from within the porous media which is being drawn downward through the media under influence of gravity.

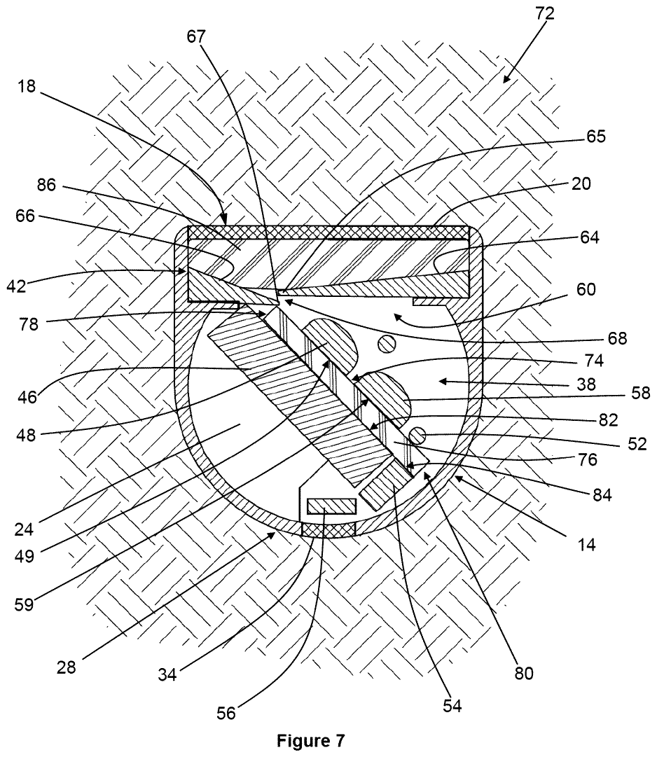

As shown in FIG. 3, the area of the inlet 18 occupies all or most or a substantial portion of an upper side of the sensor housing 14. As best illustrated with brief reference to FIG. 7, the inlet 18 is configured with an area approximately equal to a two-dimensional footprint of a sensor chamber 24 within the sensor housing 14. In the particular embodiments shown, the inlet 18 is sized to span the entire length and width of the sensor chamber 24. Returning to FIG. 3, the upper side of sensor housing 14 may be configured with only a thin bevel or edge 26 surrounding the rigid mesh 20 so as to again maximise the quantity of liquid received from the porous media through the inlet 18.

The sensor housing 14 has an inverted arch profile with a flat or planar upper side comprised substantially of the inlet 18 and a curved base 28. The sensor housing 14 includes a pair of generally planar wall portions 30 on opposite sides of the sensor housing 14 and which are connected by the curved base 28. Only one of the wall portions 30 is visible in FIG. 3. The wall portions 30 and curved base 28 are therefore parts of a single arch-shaped surface. The sensor housing 14 further includes an arch-shaped planar end wall 32 at a distal end of the sensor housing 14. The end wall 32 is selectively removable for providing access to the interior of the sensor housing 14 (i.e. the sensor chamber 24). The removability of the end wall 32 will be discussed in further detail when FIG. 6 is subsequently described.

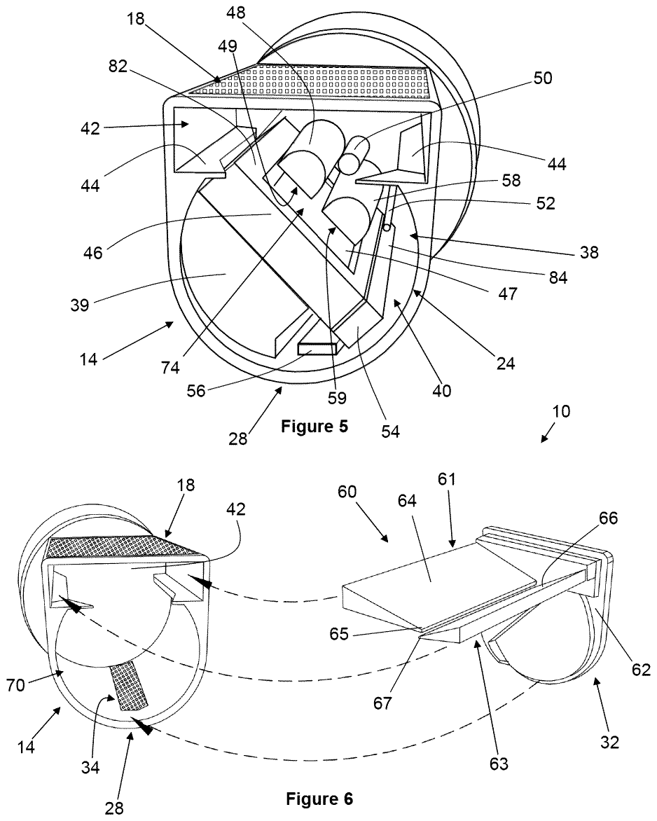

Turning to FIG. 4, the curved lower side of the sensor housing 14 includes an outlet 34. Like the inlet 18, the outlet 34 is occupied by a rigid mesh 35 which provides a series of outlet openings 36 allowing egress of liquid from the sensor housing back into the porous media. The outlet 34 has a smaller area than the inlet 18 so as to partially restrict the outflow of liquid from the sensor housing back into the porous media. Like the rigid mesh 20 at the inlet 18, the rigid mesh 35 at the outlet 34 is configured to prevent ingress of relatively large particular such as rocks, stones or clumps into the sensor housing 14. The outlet 34 has a generally elongate configuration which extends substantially the length of the sensor housing 14.

Turning to FIGS. 5 and 6, the sensor housing 14 is shown with end wall 32 removed and exposing the sensor chamber 24. The interior of the sensor housing 14 provides the sensor chamber 24 in which a sensor arrangement 38 is located. The sensor arrangement 38 is located in a sensor chamber lower portion 40 with a generally cylindrical profile. The sensor chamber 24 further includes a sensor chamber upper portion 42 directly beneath the inlet 18. The sensor chamber upper portion 42 has a generally rectangular profile. The upper portion 42 and lower portion 40 are delimited by a pair of ledges 44 extending inwardly and from opposite sides of the sensor chamber 24.

FIG. 5 illustrates the sensor arrangement 38 in position within the sensor chamber 24. The exact number, position and configuration of the sensor arrangement may vary. In the illustrated embodiment, the sensor arrangement 38 includes a plurality of sensors and, in particular, includes a pH sensor 46, a dissolved metal ion sensor 48, a temperature sensor 50, a redox (oxygen reduction potential) sensor 52, a dissolved oxygen sensor 54 and a conductivity sensor 56. The sensor arrangement further includes a solid-state reference electrode 58 such as is described in Applicant's international patent publication WO2018/201200.

The components of the sensor arrangement 38 are arranged to facilitate a flow path of liquid through the sensor housing 14 and to promote contact between the liquid flow and the sensors. The sensors may be configured to measure chemical or physical properties of the liquid. As shown in FIG. 5 (and also in FIG. 7), the pH sensor 46 is generally planar in configuration and includes an upwardly facing planar sensing surface 47 which is inclined at approximately 30° relative to the planar inlet 18 and, in use, relative to horizontal. The planar sensing surface 47 is recessed in an inclined surface 82 of the pH sensor. The pH sensor 46 is mounted via resin-bonding on an inclined support 39 which occupies a volume of the sensor chamber 24 diagonally below the pH sensor 46. The support 39 is configured to occupy a portion of the volume within the sensor chamber 24 and thereby raise the level of condensate dammed within the chamber 24. The support 39 may thereby facilitate correct operation of one or more sensors (for example the conductivity sensor 56).

The dissolved metal ion sensor 48 includes a downwardly facing planar sensing surface 49. The reference electrode 58 includes a downwardly facing planar sensing surface 59. The dissolved metal ion sensor 48 and reference electrode are spaced apart from and above the pH sensor planar sensing surface 47 so as to provide an elongated gap 74 that extends between the pH sensor 46 and the metal ion sensor and reference electrode 58. The reference electrode 58 is positioned diagonally downward of the dissolved metal ion sensor 48.

The temperature sensor 50 is configured for measurement of air temperature in the sensor chamber 50 and is therefore located generally above the reference electrode 58 and out of the liquid flow path. Measurement of the air temperature in the sensor chamber will typically be akin to the temperature of the liquid within the porous media and therefore also the temperature of the porous media (for example the temperature of a heap). As is best shown in FIG. 7, the redox sensor 52 is positioned diagonally downward of the reference electrode 58 and approximately along a diagonal axis defined by the dissolved metal ion sensor 48 and the reference electrode 58. The dissolved oxygen sensor 54 is spaced apart from and is located diagonally downwardly of a lower end of the pH sensor 46. The dissolved oxygen sensor is approximately aligned with the pH sensor along a diagonally orientated plane. In particular, the dissolved oxygen sensor 54 includes an inclined surface 84 which is approximately co-planar with the inclined surface 82 of the pH sensor 46. Both the pH sensor inclined surface 82 and the dissolved oxygen sensor inclined surface 84 are inclined at approximately 30° to the inlet 18 and, in use, to a horizontal direction.

The redox sensor 52 is spaced from the dissolved oxygen sensor 54 by approximately the same distance as the reference electrode 58 and dissolved metal ion sensor 48 are spaced from the pH sensor 46. The elongated gap 74 is thereby continued diagonally downwardly along and above the pH sensor and the dissolved oxygen sensor. The conductivity sensor 56 is positioned within the base 28 of the sensor housing and vertically above the outlet 34.

FIG. 6 illustrates an exploded view illustrating the end wall 32 removed from the sensor housing 14. The sensor arrangement 38 is not shown in FIG. 6 in order to also illustrate the internal side of the outlet 34 at the base 28 of the sensor housing 14.

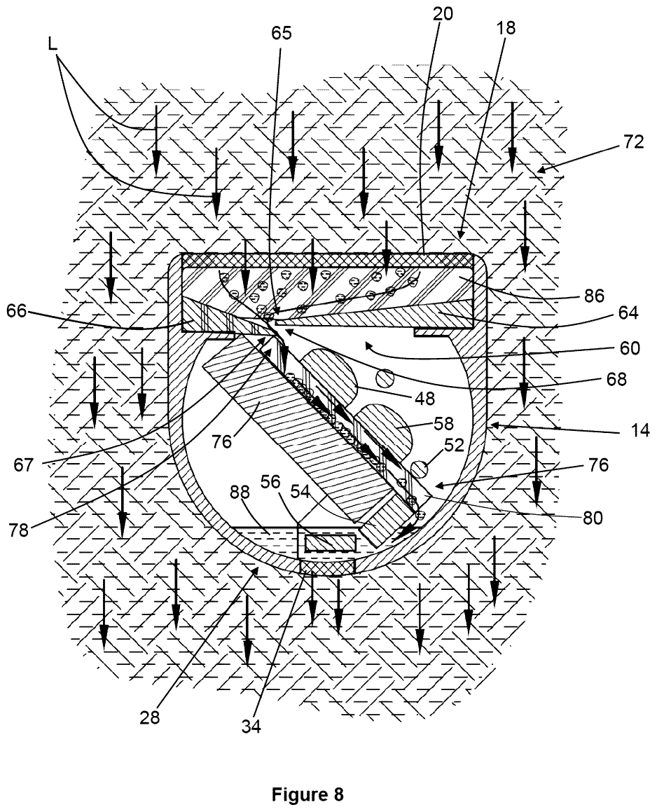

As shown in FIG. 6, the sensor apparatus 10 further includes a directing arrangement 60 extending from an inwardly facing side 62 of the removable end wall 32. The directing arrangement 60 is configured for insertion into the sensor chamber upper portion 42 when the end wall 32 is positioned at an open end 70 of the sensor housing 14. The end wall 32 is removably connected at the open end 70 by resin bonding. Alternatively, the end wall 32 could be removably connected by press fit or interference fit. The directing arrangement 60 is locatable directly below the inlet 18 in order to receive and direct liquid received through the inlet 18 along a predetermined flow path which will be described in further detail with respect to FIG. 8

Referring to FIGS. 6 and 7, the directing arrangement 60 includes a pair of triangular protrusions 61, 63 extending from the inwardly facing side 62 of the end wall 32 and which provide an upper inclined surface 64 and a lower inclined surface 66. The upper inclined surface extends downwardly until terminating at a lower edge 65. The lower inclined surface extends downwardly until terminating at a lower edge 67. The lower edge 65 of the upper inclined surface 64 is positioned at a higher level than a lower edge 67 of the lower inclined surface 66 such that liquid flow from the upper inclined surface 64 will, in use, fall onto the lower inclined surface 66 and in particular will fall near to the lower edge 67 of the lower inclined surface 66.

According to the configuration of this particular embodiment, liquid captured across the length of the upper inclined surface 64 tends to move downwardly along the upper inclined surface 64 and is directed onto the lower inclined surface 66 and then subsequentially directed by the lower edge 67 of the lower inclined surface 66 toward (for example, onto) the sensor arrangement 38. In particular, the lower edge 67 of the lower inclined surface 66 is configured to direct fluid captured by the fluid directing arrangement onto an upper part of the sensor arrangement 38 and, more particularly, an upper part of an inclined portion of the sensor arrangement 38.

FIG. 7 provides a cross sectional view of the sensor housing 14 with the sensor arrangement 38 and the directing arrangement 60 in their respective positions within the sensor chamber 24. The sensor housing 14 is shown located within a porous media 72 such as an ore heap in an industrial heap leaching process.

As shown, the upper and lower inclined surfaces 64, 66 collectively extend across an entire width of the inlet 18. A second stage filter 86 is located between the inlet 18 and the inclined surfaces 64, 66. The second stage filter 86 is therefore located directly below the first stage filter which comprises the rigid mesh 20. The second stage filter 86 may have a sponge-like structure allowing for resilient deformation and whereby the filtering material 86 may be compressed between the inlet 18 and the inclined surfaces 64, 66 so as to substantially occupy the volume of the sensor chamber upper portion 42 above inclined surfaces 64, 66.

The second stage filter 86 is positioned to receive liquid that has passed through the rigid mesh 20 at the inlet 18 which acts as a first stage filter to prevent ingress of relatively large particulates. The second stage filter 86 may act to capture medium-size particulates and remove it from the liquid flow that exits the second stage filter 86 and which is collected by the inclined surfaces 64, 66 of the directing arrangement 60. In this manner, flow which forms at or is collected by the directing arrangement 60 has been filtered by the first stage and second stage filters 20, 86 so as to remove large and medium size particulates.

The lower edges 65, 67 of the inclined surfaces 64, 66 of the directing arrangement 60 are vertically above an upper part of the sensor arrangement 38 and, in particular, above an upper end of a moistening material 76 which, in use, defines an upstream end 78 of the moistening material 76. The directing arrangement 60 is thereby configured to direct liquid onto the upstream end 78 of the moistening material 76. In particular, the directing arrangement 60 includes an opening 68 between the lower edges 65, 67 of the inclined surfaces 64, 66 through which the received liquid is funnelled. The opening 68 is positioned vertically above the upstream end 78 of the moistening material 76.

The moistening material 76 comprises a sponge-like structure configured to allow diffusion of liquid whilst also allowing for flow of liquid from the upstream end 78 to a downstream end 80 of the moistening material 76. The moistening material 76 has a generally planar or sheet-like configuration. The moistening material 76 extends through the above-noted gap 74. The moistening material 76 overlies the inclined surface 82 of the pH sensor 46 and the inclined surface 84 of the dissolved oxygen sensor 54. The moistening material 76 provides a flow passage for the received liquid. The moistening material 76 is therefore coincident with a portion of the predetermined flow path through the sensor housing 14.

As will be appreciated from FIG. 7, the sensor arrangement 38 includes sensors positioned on opposite upper and lower sides of the moistening material 76 and thereby on opposite upper and lower sides of the flow path. In particular, the metal ion sensor 48, reference electrode 58 and redox sensor 52 are positioned along an upper side of the flow path and the pH sensor 46 and dissolved oxygen sensor 54 are positioned along a lower side of the flow path. For a given number of sensors, this configuration allows for the sensor arrangement 38 to be contained within a smaller volume as compared to an alternative configuration in which the sensors are positioned in a row on the same side of the flow path.

The moistening material 76 may have a resilient configuration which enables it to be partially compressed into the gap 74. The moistening material 76 contacts a number of components within the sensor arrangement in order to maintain a moist or saturated condition for these components. The moistening material 76 thereby wets the surface of the sensors in contact with the moistening material 76. In particular, the moistening material contacts and wets the pH sensor 46 and its planar sensing surface 47 (best shown in FIG. 5). The moistening material 76 contacts and wets the planar sensing surface 49 of the dissolved metal ion sensor 48. The moistening material 76 contacts and wets the planar sensing surface 59 of the reference electrode 58. The moistening material also contacts and wets the inclined surface 84 of the dissolved oxygen sensor 54. Furthermore, the moistening material 76 contacts and wets the redox sensor 52.

The downstream end 80 (i.e. lower end) of the moistening material 76 is located generally proximate to the base 28 of the sensor housing 14 and whereby liquid exiting the downstream end 80 will be captured at an the interior of the base 28 and pool around the outlet 34 whereupon the conductivity sensor 56 is contacted by the pooled or damned liquid.

Having described above the components of the sensor housing 14, reference is now made to FIG. 8 which illustrates operation of the sensor apparatus 10 and in particular the flow of liquid from within a porous media 72 along a predetermined flow path through the sensor housing 14.

FIG. 8 illustrates a side section of the sensor apparatus 10 and, in particular, the sensor housing 14 located within a region of porous media 72 through which a liquid L is moving downwardly under the influence of gravity. The liquid passes through horizontal inlet 18 and through rigid mesh 20 within the inlet 18 which operates as a first stage filter preventing relatively large particulates from entering the sensor housing 14. The liquid received through inlet 18 passes downwardly through the second stage filter 86 located below (i.e. on the inside of) the inlet 18 which operates as a second stage filter removing from the liquid medium-sized particulates which is captured by the second stage filter 86.

The received liquid passed through the second stage filter 86 is directed by the upper and lower inclined surfaces 64, 66 of the directing arrangement 60 toward the lower edge 67 of the lower inclined surface 66. The received liquid is funnelled by the inclined surfaces 64, 66 through the opening 68 between the lower edges 65, 67 and then onto the upstream end 78 of the moistening material 76 which overlies the pH sensor 46. The received liquid is diffused through the moistening material 76 to wet the surface of sensors in contact with the moistening material 76 to enable measurement of the liquid properties. In particular, the pH sensor 46, metal ion sensor 48, reference electrode 58, redox sensor 52 and dissolved oxygen sensor 54 are all located in contact with the moistening material 76 and thereby all positioned along the flow path of the received liquid as it passes from an upstream end 78 to a downstream end 80 of the moistening material 76.

Still referring to FIG. 8, the flow path of the received liquid exits the downstream end 80 of the moistening material 76 and forms a pool 88 at the interior of the base 28 which, in the illustrated embodiment, submerges the conductivity sensor 56. It will be appreciated that the conductivity sensor 56 need not necessarily become submerged and may simply be contacted by the received liquid. It will also be appreciated that the level of liquid pool 88 at the base 28 may vary depending on the flow rate of liquid through the porous media 72. The liquid pool 88 is located above the outlet 34 such that liquid flows from the pool 88 through the outlet 34 in order to egress the sensor chamber 24 and return to the porous media 72.

The size of the outlet 34 (particularly its size relative to the inlet 18) could be selected based on an intended application and/or anticipated liquid flow rate through the porous media 72. The size of the outlet 34 may be selected to provide a desired restriction of flow egress so as to provide or maintain a desired level of liquid pool 88 within the base 28. For example, in an application with an anticipated porous media liquid flow rate that is relatively low, the outlet 34 might be configured with a smaller size than that which is shown in the illustrated embodiment. Similarly, for use in porous media with an anticipated higher liquid flow rate, the outlet 34 may be provided with larger area so as to increase the outlet rate.

In particular applications, the outlet rate may initially be less than the inlet rate but the outlet rate may increase as liquid becomes pooled or damned at the base of the sensor housing. The inlet flow may vary with time depending on the irrigation pattern applied to the top of an ore heap and also due to a breakdown of ore over time which affects liquid flow through the medium.

In particular embodiments (not illustrated) the outlet may be configured with a variable aperture to allow adjustment of the outlet size and in order to allow selective configuration of the apparatus for use with the anticipated liquid flow rate of a particular application.

Turning to FIG. 9, the apparatus 10 is shown in use with a column leaching apparatus 11 which includes the column 12 previously shown in FIGS. 1 and 2. The interior of column 12 is occupied with porous media 90. The column leaching apparatus includes a gas supply conduit 92 used to inject gases such as air, O₂, CO₂ or N₂ into the base of the column 12 to assist with leaching kinetics.

A liquid L is irrigated onto the top of the porous media and flows downwardly through the column 12 under influence of gravity. The sensor housing 14 is located within the porous media 90 and located to receive a sample of the liquid L through the inlet 18 which is orientated horizontally within the porous media 90. The sampled liquid flows through the sensor housing 14 along the predetermined flow path discussed in the foregoing in order for the sensor arrangement (not visible in FIG. 9) to acquire data representative of the liquid L.

As noted with reference to FIG. 2, a portion 13 of the sensor apparatus 11 remains outside of the column 12 and includes an electrical componentry housing 15 which contains one or more PCBs configured to receive electrical signals from the sensors and/or to transmit the sensor data to a connected receiving hub via a wired or wireless connection. The sensor chamber within the sensor housing 12 is fluidly isolated from the electrical componentry within the electrical componentry chamber 15.

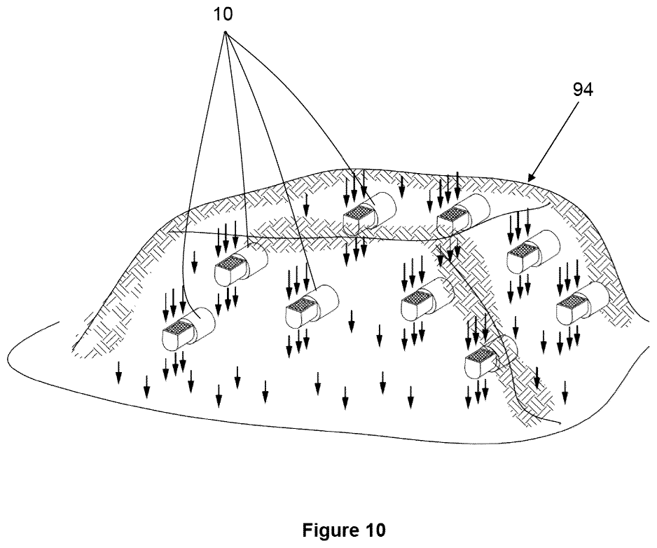

FIG. 10 exemplifies a plurality of the sensor apparatus 10 distributed throughout various regions of a bulk heap 94 in an industrial heap leaching process. The plurality of sensor apparatus 10 are submerged within the bulk heap 94. The plurality of sensor apparatuses 10 may be connected via data link cables to one another and/or to a receiving hub configured to receive data acquired about the liquid sampled by each sensor apparatus. Alternatively, the plurality of sensor apparatus may be configured for a wireless connection to a suitable receiving hub. The plurality of sensor apparatuses 10 may thereby be used to acquire in-situ data about different parts of the bulk heap 94 and, for example, data on various depths of the bulk heap 94. The data on a two-dimensional or three-dimensional spatial arrangement may be collected in the manner as described in the Applicant's earlier international patent publication WO2018/068087.

Those skilled in the art will appreciate that the invention described herein is susceptible to variations and modifications other than those specifically described. It is understood that the invention includes all such variations and modifications which fall within the spirit and scope of the present invention.

Where any or all of the terms “include”, “includes”, “included” or “including” are used in this specification (including the claims) they are to be interpreted as specifying the presence of the stated features, integers, steps or components, but not precluding the presence of one or more other features, integers, steps or components.

Where the terms “comprise”, “comprises” and “comprising” are used in the specification (including the claims) they are to be interpreted as specifying the stated features, integers, steps or components, but not precluding the presence of one or more other features, integers, steps or components, or group thereof.

1. A sensor apparatus locatable within a region of porous media for acquiring data from a liquid within the porous media, the apparatus including:
a sensor housing including an inlet for receiving liquid from the porous media into the sensor housing and an outlet for received liquid to egress the sensor housing, the apparatus configured for received liquid to flow through the sensor housing along a predetermined flow path between the inlet and the outlet;
a sensor arrangement within the sensor housing for acquiring data from the flow of liquid between the inlet and the outlet, the sensor arrangement including one or more sensors positioned along the flow path for contacting the liquid and configured for measuring one or more properties of the liquid to acquire data representative of the liquid at the porous media region; and
a filtration arrangement configured for removing particulates from the liquid prior to contacting the one or more sensors.

2. An apparatus according to claim 1, wherein the apparatus is configured for location within a partially saturated porous media region and for forming a locally saturated condition for at least one sensor of the sensor arrangement to facilitate data acquisition by the sensor arrangement.

3. (canceled)

4. An apparatus according to claim 1, the apparatus including a directing arrangement configured to direct liquid received through the inlet toward the sensor arrangement.

5. An apparatus according to claim 4, the directing arrangement located between the inlet and the sensor arrangement and configured to funnel liquid collected from the inlet toward the sensor arrangement.

6. An apparatus according to claim 4, the directing arrangement including at least one inclined surface extending below the inlet and terminating at a lower edge orientated toward the sensor arrangement and wherein, in use, the liquid flow is collected or formed on the inclined surface and flows toward the lower edge and from the lower edge toward the sensor arrangement.

7. (canceled)

8. (canceled)

9. (canceled)

10. An apparatus according to claim 1, including a moistening material in contact with the one or more sensors of the sensor arrangement, the moistening material positioned within the flow path and configured to maintain a moist or saturated condition at the one or more sensors to facilitate measurement of one or more properties of the liquid by the one or more sensors.

11. An apparatus according to claim 10, the sensor arrangement including a plurality of sensors in contact with the moistening material and wherein at least one sensor contacts a first side of the moistening material and at least one sensor contacts a second and opposite side of the moistening material.

12. (canceled)

13. (canceled)

14. (canceled)

15. (canceled)

16. An apparatus according to claim 15, the sensor arrangement including a pH sensor configured for measuring pH of the received liquid, the pH sensor positioned along a lower side of the flow path.

17. An apparatus according to claim 16, the pH sensor including a sensing surface having an inclined orientation configured for the liquid flow to move over the sensing surface.

18. (canceled)

19. (canceled)

20. (canceled)

21. (canceled)

22. An apparatus according to claim 1, the sensor arrangement including a solid-state reference electrode and wherein one or more sensors of the sensor arrangement are associated with the solid-state reference electrode.

23. An apparatus according to claim 1, the sensor arrangement including at least one sensor located out of contact with the liquid.

24. An apparatus according to claim 1, wherein the filtration arrangement includes a first stage filter located at the inlet and configured to prevent ingress of relatively large particulates from the porous media.

25. (canceled)

26. An apparatus according to claim 24, the filtration arrangement including a second stage filter configured to filter material which has passed through the first stage filter and to remove smaller-sized particulates than the first stage filter.

27. (canceled)

28. (canceled)

29. (canceled)

30. An apparatus according to claim 24 the filtration arrangement including a third stage filter for filtering liquid which has passed through the second stage filter and to remove smaller-sized particulates than the second stage filter.

31. (canceled)

32. An apparatus according to claim 30, the second and/or third stage filters having anti-fouling properties to reduce or resist biofouling at the sensor arrangement.

33. (canceled)

34. (canceled)

35. (canceled)

36. (canceled)

37. An apparatus according to claim 1 wherein the sensor arrangement includes one or more of a pH sensor, a dissolved metal ion sensor, a temperature sensor, a redox sensor, a dissolved oxygen sensor or a conductivity sensor.

38. (canceled)

39. (canceled)

40. (canceled)

41. (canceled)

42. (canceled)

43. (canceled)

44. An apparatus according to claim 30, wherein the second and/or third stage filter may include a positively charged sponge, or a hydrophobic sponge, or a filter material coated with specific chemical functional groups for chemisorption or chelating of particular particulates, or a filter material coated or loaded with a specific surfactant or flocculant, or filter material or aid which aids or promotes the aggregation of ultrafine particles, for capture, or a filter material having a particular morphology (such as sponge particle shape), porosity, or surface area that promotes entrapment or capture or attraction of particular particulates of specific shape or size or molecular structure.

45. A column leaching apparatus including an upright column having a peripheral wall which includes an opening and the column leaching apparatus further including a sensor apparatus according to claim 1, wherein the sensor apparatus extends through the opening in the peripheral wall and into an interior of the column.

46. A system for acquiring data from liquid in a heap leaching application including a plurality of sensor apparatuses according to claim 1 to wherein the sensor apparatuses are distributed throughout regions of heap leaching material to be analysed and the sensor apparatus configured to acquire and transmit sensor data about the liquid within the region of each sensor apparatus to one or more hubs.

47. A method of acquiring data from liquid within a porous media to be analysed including the steps of locating one or more sensor apparatuses according to claim 1 within the porous media, the one or more sensor apparatuses being configured periodically acquire sensor data relating to chemical or physical properties of the fluid surrounding the sensor over a predetermined period of time and to periodically transmit the sensor data via a data link to one or more hubs.
